(19) United States
(12) Patent Application Publication
(10) Pub. No.: US 2024/0249834 A1
(43) Pub. Date: Jul. 25, 2024

(54) BIOMETRIC MONITORING OVER A WIRELESS INTERCOM SYSTEM

(71) Applicant: David Clark Company Incorporated, Worcester, MA (US)

(72) Inventors: Eric Motler, Northborough, MA (US); Brad Deschenes, Dudley, MA (US)

(73) Assignee: David Clark Company Incorporated, Worcester, MA (US)

(21) Appl. No.: 18/100,272

(22) Filed: Jan. 23, 2023

(51) Int. Cl. G16H 40/63

(52) U.S. Cl. CPC G16H 40/63

(57) ABSTRACT

Systems and methods are provided for updating data in a computer network. An exemplary method includes: receiving, using a first communication medium, sensor data from one or more sensors at a first computing device; processing the sensor data from each of the one or more sensors to determine biometric data of at least one user; sending the biometric data to a second computing device for processing resulting in second data, wherein the second computing device comprises a DECT-based communication medium for communicating with other computing devices; determining a location to which the second data is to be sent; and upon determining the location to which the second data is to be sent, routing, using the DECT-based communication medium, the second data to the location, wherein the first communication medium is different from the DECT-based communication medium.

### BACKGROUND

Biometrics are becoming very prevalent in personal smart devices in the market today. Sensor technology and the number of non-invasive sensors and what they can measure is growing rapidly. They can provide local feedback to the user about vitals or other meaningful health related metrics.

However, sharing those vitals typically requires a cellular connection, Internet connection, or someone co-located looking at the same reporting device as the user. Alerts and warnings for when those metrics fall to unsafe levels are typically reported to the user locally, or perhaps an audible warning could be heard by others near and in a quiet environment. If there is no cellular connection, Internet connection, or someone co-located looking at the same reporting device as the user, the capability of sharing these vitals is nearly impractical.

There is a need to provide a system for biometric monitoring over an intercom system.

### SUMMARY

According to one aspect of the subject matter described in this disclosure, a method for biometric monitoring is provided. The method includes: receiving, using a first communication medium, sensor data from one or more sensors; processing the sensor data from each of the one or more sensors to determine biometric data of at least one user; determining a location to which the biometric data is to be sent; and upon determining the location to which the biometric data is to be sent, routing, using a DECT-based communication medium, the biometric data to the location, wherein the first communication medium is different from the DECT-based communication medium.

According to another aspect of the subject matter described in this disclosure a method for biometric monitoring is provided. The method includes the following: receiving, using a first communication medium, sensor data from one or more sensors at a first computing device; wherein the first computing device comprises a DECT-based communication medium for communicating with other computing devices; processing the sensor data from each of the one or more sensors to determine biometric data of at least one user; determining whether auxiliary processing of the biometric data is required; and upon determining whether auxiliary processing of the biometric data is required, routing, using a DECT-based communication medium, the biometric data to an auxiliary location for performing the auxiliary processing of the biometric data by a second computing device, wherein the first communication medium is different from the DECT-based communication medium.

According to another aspect of the subject matter described in this disclosure, a system for biometric monitoring is provided. The system includes one or more computing device processors, and one or more computing device memories. The one or more computing device memories are coupled to the one or more computing device processors. The one or more computing device memories storing instructions executed by the one or more computing device processors. The instructions are configured to: receive, using a first communication medium, sensor data from one or more sensors at a first computing device; process the sensor data from each of the one or more sensors to determine biometric data of at least one user; send the biometric data to a second computing device for processing resulting in second data, wherein the second computing device comprises a DECT-based communication medium for communicating with other computing devices; determine a location to which the second data is to be sent; and upon determining the location to which the second data is to be sent, routing, using the DECT-based communication medium, the second data to the location, wherein the first communication medium is different from the DECT-based communication medium.

According to another aspect of the disclosure, a non-transitory computer-readable storage medium storing instructions which when executed by a computer cause the computer to perform method for biometric monitoring is provided. The method includes the following: receiving, using a first communication medium, sensor data from one or more sensors at a first computing device; processing the sensor data from each of the one or more sensors to determine biometric data of at least one user; sending the biometric data to a second computing device for processing resulting in second data, wherein the second computing device comprises a DECT-based communication medium for communicating with other computing devices; determining a location to which the second data is to be sent; and upon determining the location to which the second data is to be sent, routing, using the DECT-based communication medium, the second data to the location, wherein the first communication medium is different from the DECT-based communication medium.

Additional features and advantages of the present disclosure is described in, and will be apparent from, the detailed description of this disclosure.

### BRIEF DESCRIPTION OF THE DRAWINGS

The disclosure is illustrated by way of example, and not by way of limitation, in the figures of the accompanying drawings in which like reference numerals are used to refer to similar elements. It is emphasized that various features may not be drawn to scale and the dimensions of various features may be arbitrarily increased or reduced for clarity of discussion.

FIG. 1 is a schematic diagram of an wireless intercom network 100, in accordance with some embodiments.

FIG. 2 is a schematic diagram of an exemplary endpoint device of the wireless intercom network of FIG. 1, in accordance with some embodiments.

FIG. 3 is a schematic diagram of an exemplary embodiment of a master station, in accordance with some embodiments.

FIG. 4 is a process flowgraph of operations included in an example process for monitoring biometric data, in accordance with some embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context.

This disclosure describes a biometric monitoring system and methods for biometric monitoring over a wireless intercom system. The biometric monitoring system bypasses cellular or Internet connections to communicate biometric data to users and/or systems. The biometric monitoring system may utilize the Digital Enhanced Cordless Telecommunications (DECT) communication protocol to establish a wireless network for communicating between various devices. Moreover, the biometric monitoring system uses a star topology where a master station receives data from several endpoint devices. Each endpoint device is connected to at least one sensor attached to a specific user(s). The sensors send sensor data to their respective connected devices. Each device processes its sensor data to produce biometric data of the particular user(s). The master station processes the biometric data and sends indications to the devices indicating unsafe conditions associated with the specific user or a vicinity. Moreover, the endpoint devices could also process data and alert locally.

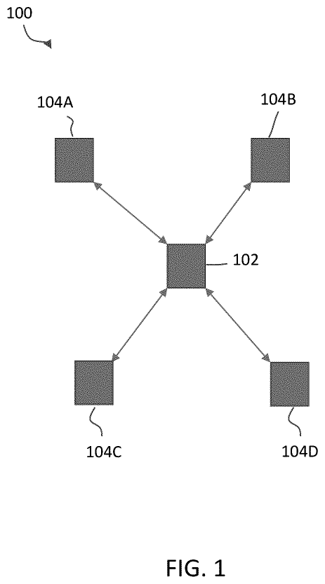

FIG. 1 is a schematic diagram of a wireless intercom network 100, in accordance with some embodiments. The wireless intercom network 100 is arranged in a star topology where a master station 102 is connected to several endpoint devices 104A-104D. Master station 102 and endpoint devices 104A-104D are equipped with communication applications for communicating wirelessly using the DECT standard. Moreover, DECT is designed for short-range usage, which acts as an access method to various networks. In addition, DECT eliminates the need for master station 102 and endpoint devices 104A-104D to use a cellular or Internet connection to establish wireless communications.

In some embodiments, the wireless intercom network 100 may be implemented on a digital intercom, a combination of DECT and Ethernet wired devices. This architecture may have the same star topology, but the medium in which data could be exchanged between master station 102 and endpoint devices 104 may be wired Ethernet (non-DECT).

Each device 104A-104D sends wirelessly biometric data to master station 102. Moreover, master station 102 analyzes and monitors the biometric data to determine if the user or vicinity associated with endpoint devices 104A-104D is experiencing unsafe conditions. For example, once a determination is made that device 104A is experiencing an unsafe condition, master station 102 may send a trigger instruction to device 104A that an unsafe condition is present. There may be different trigger instructions for different unsafe conditions. The trigger instruction may initiate an alert to be set off locally at a device 104A-104D. The unsafe conditions may include:

Dangerous health indicator(s) for the specific user(s).

Detecting unhealthy gases or fluids.

Other detectable dangerous events in the vicinity of devices 104A-104D.

In some embodiments, each endpoint device 104A-104D may display a message sent in response to a trigger instruction from master station 102, indicating an unsafe condition is present. In some embodiments, the trigger instruction may initiate an audio message to be played at an endpoint device 104A-104D, indicating an unsafe condition is present.

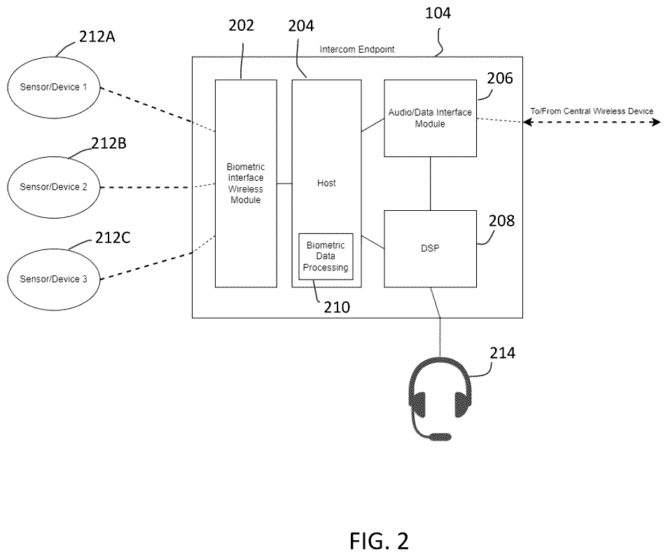

FIG. 2 is a schematic diagram of an exemplary endpoint device 104 of the wireless intercom network 100, in accordance with some embodiments. The exemplary endpoint device 104 may be an exemplary embodiment of any endpoint device 104A-104D of the wireless intercom network 100. In particular, endpoint device 104 includes a biometric interface wireless module 202 that is wirelessly connected to sensors 212A-212C. In this exemplary embodiment, sensors 212A-212C may be wearable sensors that acquire information for a user(s) or a vicinity close to the user. The data each sensor 212A-212C acquires from the user(s) are processed as sensor data and sent to the biometric interface wireless module 202. In some instances, the sensor data may include the operational status of sensors 212A-212C.

In some embodiment, sensors 212A-212C may be attached to the body of a user(s) to acquire biometric data. In some embodiment, sensors 212A-212C may detect gases or other volatiles deemed dangerous around the user's vicinity.

The sensors 212A-212C may communicate wirelessly with biometric interface wireless module 202 to send their sensor data. The biometric interface wireless module 202 acts as an interface by processing the sensor data received into processed sensor data useable for further processing in exemplary endpoint device 104. The biometric interface wireless module 202 sends the processed sensor data to host system 204. The biometric interface wireless module 202 may use Bluetooth for communicating wirelessly with sensors 212A-212C. However, other wireless communication protocols may be used to establish wireless communications between sensors 212A-212C and biometric interface wireless module 202.

The host system 204 controls and manages the overall operations of exemplary endpoint device 104. One of the critical operations performed by host system 204 is processing the processed sensor data to produce biometric data for each sensor 212A-212C. In particular, host system 204 uses biometric data processing module 210 to create biometric data from the processed sensor data. The biometric data processing module 210 may be a collection of applications and libraries referenced by host system 204 to produce the biometric data.

Also, the host system 204 may issue a local alert to a local user based on biometric data processing outcome of biometric data processing module 210. In some instances, the local alert may be an audio alert that is played on exemplary endpoint device 104 or via headset 214. The local alert may be a visual alert to the local user and others in proximity, such as a LED or some other visual indicators. Moreover, the local alert may be haptic to the local user, such as vibration motor in the belt pack/headset 214.

Moreover, the host system 204 may control the operations of audio/data interface module 206 and digital signal processor (DSP) 208. The audio/data interface module 206 acts as an interface for receiving and sending wireless data between master station 102 and exemplary endpoint device 104. In particular, the wireless intercom network 100 may be used to establish wireless communications between master station 102 and exemplary endpoint device 104 using the wireless intercom network 100. Using DECT, the master station 102 and exemplary endpoint device 104 do not require a cellular or Internet connection to establish a wireless connection.

Exemplary endpoint device 104 and master station 102 use a wireless DECT connection to send wireless data to each other. In particular, the host system 204 uses a wireless DECT connection to transmit biometric data to master station 102 for further processing. The audio/data interface module 206 encodes the biometric data into wireless data for sending to master station 102. Moreover, host system 204 may instruct audio/data interface module 206 to send status data of endpoint device 104 to master station 102 using the same approach for sending biometric data to master station 102.

The wireless data is most compatible with wireless transmission because it is formulated in accordance with the DECT standard, including data packet size and other criteria. The wireless intercom network 100 is arranged in a star topology where a master station 102 is connected to several endpoint devices 104A-104D. In addition, DECT eliminates the need for master station 102 and endpoint device 104 to use a cellular or Internet connection to establish wireless communications in wireless intercom network 102.

Audio/data interface module 206 is equipped with communication applications for encoding and decoding wireless communications between master station 102 using DECT. Wireless data sent to exemplary endpoint device 104 by master station 102 is first received by audio/data interface module 206. The audio/data interface module 206 decodes the wireless data to retrieve from the wireless data the actual data sent by master station 102. At this point, the host system 204 may direct audio/data interface module 206 to send the actual data to host system 204 for further processing.

Audio/data interface module 206 may include location information of other endpoint devices 104 in intercom network 100, including master station 102. Host system 204 may store the location information of each endpoint device 104 in wireless intercom network 102 in its memory. When host system 204 determines to send biometric data to master station 102, audio/data interface module 206 can retrieve the location information of master station 102 from the host system 204. In some embodiments, audio/data interface module 206 may send data to other endpoint devices in wireless intercom network 100 using the stored locations of different endpoint devices 104.

The location information of an endpoint 104 may be generated locally and sent to master station 102 using the audio/data interface module 206 of endpoint device 104. The master station 102 may also send its location information and the location information of other endpoint devices to endpoint device 104. This may allow an endpoint device 104 to know the locations of other endpoint devices and master station 102 to assist when in need. When an endpoint device 104 cannot send to other endpoint devices directly, one may use the master station 102. In this case, endpoint device 104 sends data to master station 102, and master station 102 sends the data to the other endpoint devices.

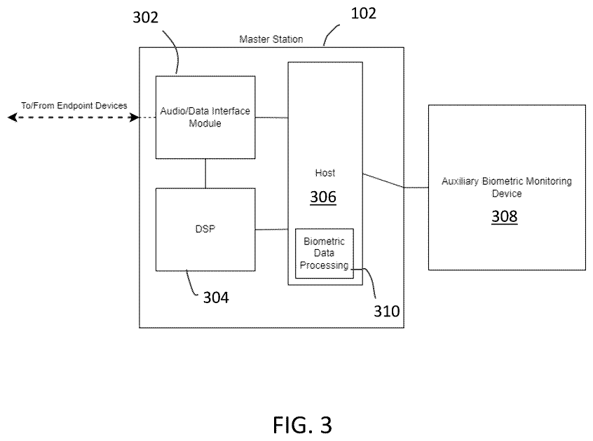

There may be certain circumstances where displaying biometric data is necessary, the master station 102 uses auxiliary biometric monitoring device 308 to display biometric data of the intercom users, as shown in FIG. 3. In this case, the host system 204 may direct audio/data interface module 206 to request master station 102 if the biometric data is sent directly to the auxiliary biometric monitoring device 308. The details of the operations of auxiliary biometric monitoring device 308 are described further below.

Moreover, host system 204 may initiate playing of an audio message as an alert when a trigger instruction is received by an endpoint device 104 from master station 102. When this occurs, host system 204 instructs DSP 208 to play the audio message. The host system 204 may direct DSP 208 to send the audio data to headphone 214 to be heard by the user(s). The audio message may be stored locally in an endpoint device 104. In addition, the host system 204 may initiate audio messages for other interactional purposes besides an alert.

Host system 204 includes one or more processing units for executing the various applications used in its operations. Moreover, host system 204 includes one or more memory systems for storing data, including biometric and application data.

FIG. 3 is a schematic diagram of an exemplary embodiment of master station 102 of FIG. 1, in accordance with some embodiments. In particular, the master station 102 includes an audio/data interface module 302, DSP 304, and a host system 306. As shown in FIG. 1, the master station 102 wirelessly communicates with endpoint devices 104A-104C in wireless intercom network 100 using DECT. Both master station 102 and endpoint devices 104A-104D transmit and receive wireless data from each other. When sending biometric data to master station 102, endpoint devices 104A-104D encode the biometric data into wireless data for transmission using the DECT standard, as described herein.

Host system 306 controls the operations of audio/data interface module 302 and DSP 304. Specifically, host system 306 directs how wireless data received from endpoint devices 104A-104D are processed once received at master station 102. Also, host system 306 directs the data that master station 102 sends to endpoint device 104A-104D, including alerts regarding unsafe conditions.

The audio/data interface module 302 receives wireless data from endpoint devices 104A-104D. Afterward, the audio/data interface module 302 decodes the received wireless data to retrieve the relevant data. The relevant data may be biometric data, audio signals, status of the sensors, status of an endpoint device, or other data used in the additional processing of the biometric data.

To process biometric data, host system 306 uses biometric processing module 310 to analyze the biometric data. Host system 306 may actively monitor biometric data for unsafe conditions. In some instances, host system 306 may allow auxiliary biometric monitoring device 308 to actively display the biometric data received from endpoint devices 104A-104D to determine user status at a glance. This may be based on the processing capability of master station 102 for monitoring biometric data or the overall workload of the master station 102. As described herein, the master station 102 may notify an endpoint device 104 to send the biometric data directly to auxiliary biometric monitoring device 308. Biometric monitoring device 308 may be a device like a display monitor showing a graphical representation of biometric summary and location of the intercom users at endpoint devices 104. This would allow someone collocated with the master station 102 to strategically intervene and instruct users of intercoms of the status of users.

When unsafe conditions are detected in the biometric data, biometric processing module 310 notifies host system 306. Host system 306 directs audio/data interface module 302 to send one or more trigger instructions to one or more specific endpoint devices 104A-104D to which the alert is relevant. The audio/data interface module 302 encodes the trigger instructions into wireless data for transmission to one or more specific endpoint devices 104A-104D. Also, host system 306 may instruct audio/data interface module 302 to notify other endpoint devices regarding sending a trigger instruction. This include sending location information of the affected endpoint device(s) to the remaining endpoint devices.

Also, the host system 306 may issue a local alert to anyone at master station 102 based on biometric data processing outcome of biometric data processing module 310. Moreover, the host system 306 may issue a local alert to anyone at master station 102 based on the status of any endpoint device 104. In some instances, the local alert may be an audio alert that is played on master station 102 via a speaker connected to master station 102 or biometric monitoring device 308. The local alert may be a visual alert to anyone in proximity of master station 102, such as LED indicators or biometric monitoring device 308.

Endpoint devices 104A-104D may send video/audio signals to master station 102. In this case, the endpoint devices 104A-104D may encode the video/audio signals into wireless data for transmission to master station 102. Audio/data interface module 302 receives and decodes this wireless data to acquire the video/audio signals. Host system 306 may direct audio/data interface module 302 to send the video/audio signals to DSP 304. The DSP 304 processes the video/audio signals into video/audio data compatible with viewing or hearing on host system 306.

Host system 306 includes one or more processing units for executing the various applications used in its operations. Moreover, host system 306 includes one or more memory systems for storing data, including biometric and application data. Also, host system 306 includes one or more audio systems for playing audio and one or more display systems for visualizing information, such as biometric data or the like.

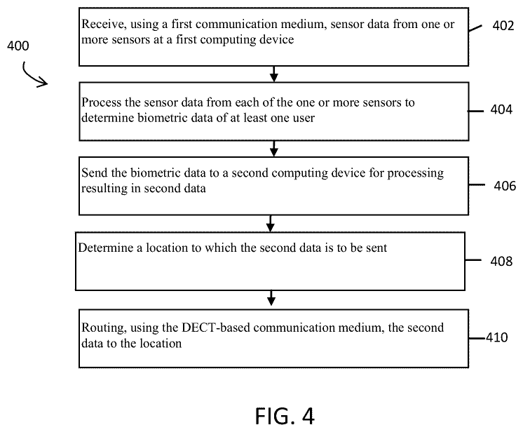

FIG. 4 is a process flowgraph of operations included in an example process 400 for biometric monitoring, in accordance with some embodiments. The operations may be implemented using computer-executable instructions stored on one or more non-transitory machine-readable storage media. The instructions may be executed by one or more processing devices, such as the host system 204 and/or host system 306, as described in FIGS. 2 and 3, to implement the operations.

Process 400 includes receiving, using a first communication medium (such as biometric interface wireless module 202), sensor data from one or more sensors (such as sensors 212A-212C) using a first computing device (endpoint device 104) (Step 402). Process 400 includes processing the sensor data from each of the one or more sensors to determine biometric data of at least one user (Step 404). The biometric data is sent to a second computing device (such as master station 102) for processing resulting in second data (such as trigger instructions) (Step 406). The second computing device includes a DECT-based communication medium (such as audio/data interface module 206 or 302) for communicating with other computing devices. A location (such as location information) is identified to which the second data is to be sent (Step 408). Upon determining the location to which the second data is to be sent, process 400 includes routing, using a DECT-based communication medium, the second data to the location, wherein the first communication medium is different from the DECT-based communication medium (Step 410).

In some embodiments, the one or more sensors may be wearable sensors.

In some embodiments, the biometric data may include information involving at least one dangerous health indicator for the at least one user. In some embodiments, the biometric data may include information associated with detectable dangerous events in a vicinity of at least one of the one or more sensors.

In some embodiments, the first communication medium is configured to have Bluetooth to communicate with the one or more sensors. In some embodiments, the DECT communication medium may identify a network location of a processing system configured to receive and process the biometric data from the DECT communication medium. In some embodiments, the processing system may be configured to be a member of a wireless intercom network.

The disclosure describes an architecture for positive identification and verification of multi-mode frequency hopping signals. The advantages provided by the architecture include the positive identification of a device in an unknown operational mode with over 100 possibilities. The architecture utilizes a Doppler mitigation technique to enable accurate demodulation in the presence of a frequency offset. Also, the architecture includes a hierarchy of verification methods for the fastest feasible identification. Moreover, the architecture allows for compensating measurement errors in timing profiles over long durations and measuring timing profiles without knowing if TDD is present or not.

Reference in the specification to “one implementation” or “an implementation” means that a particular feature, structure, or characteristic described in connection with the implementation is included in at least one implementation of the disclosure. The appearances of the phrase “in one implementation,” “in some implementations,” “in one instance,” “in some instances,” “in one case,” “in some cases,” “in one embodiment,” “in some exemplary embodiments,” or “in some embodiments” in various places in the specification are not necessarily all referring to the same implementation or embodiment.

Finally, the above descriptions of the implementations of the present disclosure have been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Accordingly, the present disclosure is intended to be illustrative, but not limiting, of the scope of the present disclosure, which is set forth in the following claims.

What is claimed is:

1. A method for biometric monitoring, the method comprising:
receiving, using a first communication medium, sensor data from one or more sensors at a first computing device;
processing the sensor data from each of the one or more sensors to determine biometric data of at least one user;
sending the biometric data to a second computing device for processing resulting in second data, wherein the second computing device comprises a DECT-based communication medium for communicating with other computing devices;
determining a location to which the second data is to be sent; and
upon determining the location to which the second data is to be sent, routing, using the DECT-based communication medium, the second data to the location, wherein the first communication medium is different from the DECT-based communication medium.

2. The method of claim 1, wherein the one or more sensors are wearable sensors.

3. The method of claim 1, wherein the biometric data comprises information involving at least one dangerous health indicator for the at least one user.

4. The method of claim 1, wherein the biometric data comprises information associated with detectable dangerous events in a vicinity of at least one of the one or more sensors.

5. The method of claim 1, wherein the first communication medium comprises Bluetooth for communicating with the one or more sensors.

6. The method of claim 1, wherein determining the location comprises the identifying, from the second computing device, a network location of a computing device configured to receive and process the second data.

7. The method of claim 1, wherein the first computing system and the second computing are configured to be members of a wireless intercom network.

8. A method for biometric monitoring, the method comprising:
receiving, using a first communication medium, sensor data from one or more sensors at a first computing device; wherein the first computing device comprises a DECT-based communication medium for communicating with other computing devices
processing the sensor data from each of the one or more sensors to determine biometric data of at least one user;
determining whether auxiliary processing of the biometric data is required; and
upon determining whether auxiliary processing of the biometric data is required, routing, using a DECT-based communication medium, the biometric data to an auxiliary location for performing the auxiliary processing of the biometric data by a second computing device, wherein the first communication medium is different from the DECT-based communication medium.

9. The method of claim 8, wherein the one or more sensors are wearable sensors.

10. The method of claim 8, wherein the biometric data comprises information involving at least one dangerous health indicator for at least one use.

11. The method of claim 8, wherein the biometric data comprises information associated with detectable dangerous events in a vicinity of at least one of the one or more sensors.

12. The method of claim 8, wherein the first communication medium is configured to have Bluetooth for wirelessly communicating with the one or more sensors.

13. The method of claim 8, wherein determining whether auxiliary processing of the biometric data is required comprises identifying, using the first computing device, an auxiliary location of a processing system configured to receive and process the biometric data.

14. The method of claim 8, wherein the first computing system and the second computing device are configured to be member of a wireless intercom network.

15. A system for biometric monitoring, the system comprising:
one or more computing device processors; and
one or more computing device memories, coupled to the one or more computing device processors, the one or more computing device memories storing instructions executed by the one or more computing device processors, wherein the instructions are configured to:
receive, using a first communication medium, sensor data from one or more sensors at a first computing device;
process the sensor data from each of the one or more sensors to determine biometric data of at least one user;
send the biometric data to a second computing device for processing resulting in second data, wherein the second computing device comprises a DECT-based communication medium for communicating with other computing devices;
determine a location to which the second data is to be sent; and
upon determining the location to which the second data is to be sent, routing, using the DECT-based communication medium, the second data to the location, wherein the first communication medium is different from the DECT-based communication medium.

16. The system of claim 15, wherein the one or more sensors are wearable sensors.

17. The system of claim 15, wherein the biometric data comprises information involving at least one dangerous health indicator for the at least one user.

18. The system of claim 15, wherein the biometric data comprises information associated with detectable dangerous events in a vicinity of the one or more sensors.

19. The system of claim 15, wherein the first communication medium is configured to have Bluetooth to communicate with the one or more sensors.

20. The system of claim 15, wherein the second computing device identifies a network location of a computing device configured to receive and process the biometric data from the DECT communication medium.

21. The system of claim 15, wherein the processing system is configured to be a member of a wireless intercom network.

22. A non-transitory computer-readable storage medium storing instructions which when executed by a computer cause the computer to perform method for biometric monitoring, the method comprising:
receiving, using a first communication medium, sensor data from one or more sensors at a first computing device;
processing the sensor data from each of the one or more sensors to determine biometric data of at least one user;
sending the biometric data to a second computing device for processing resulting in second data, wherein the second computing device comprises a DECT-based communication medium for communicating with other computing devices;
determining a location to which the second data is to be sent; and
upon determining the location to which the second data is to be sent, routing, using the DECT-based communication medium, the second data to the location, wherein the first communication medium is different from the DECT-based communication medium.
